(19) United States
(12) Patent Application Publication
(10) Pub. No.: US 2022/0361489 A1
(43) Pub. Date: Nov. 17, 2022

(54) Nanopatterned Antimicrobial Surface

(71) Applicants: ECOLE POLYTECHNIQUE FEDERALE DE LAUSANNE (EPFL), Lausanne (CH); FUNDACIÓ EURECAT, Cerdanyola del Vallès (ES); CONSEJO NACIONAL DE INVESTIGACIONES CIENTIFICAS Y TÉCNICAS, Ciudad Autonoma de Buenos Aires (AR)

(72) Inventors: Magalí Lingenfelder, Lausanne (CH); Claudia Roxana Arbeitman, Buenos Aires (AR); Claudia Delgado Simão, Barcelona (ES)

(21) Appl. No.: 17/762,756

(22) PCT Filed: Oct. 6, 2020
(86) PCT No.: PCT/IB2020/059378

§ 371 Date: Mar. 23, 2022

(30) Foreign Application Priority Data
Oct. 10, 2019 (IB) PCT/IB2019/058650

(51) Int. Cl. A01N 25/34; A61L 29/08

(52) U.S. Cl. CPC A01N 25/34; A61L 29/085; A61L 2300/404; A61L 2300/608; A61L 2420/08

(57) ABSTRACT

The invention relates to an antimicrobial surface comprising a first array of nano/micro structures spaced apart on a support, characterized in that it comprises a second array of spaced apart nanostructures located on said support and on said first array nanostructures, said second array nanostructures having different size features compared to the nanostructures of said first array.

### TECHNICAL FIELD

The invention relates to medical devices and more precisely to antimicrobial surfaces to be included into medical devices.

### BACKGROUND ART

Nosocomial infections are a problem of particular importance due to their frequency, morbidity and mortality that together with the increase of bacterial resistance to antimicrobial drugs result in increased hospital stay and increased health care costs.

A subcategory of health care-associated infections is related to medical devices. Within these, the use of intravascular catheters is associated with a risk of microbial colonization and infections started at catheters are among the most frequent. In addition, catheter related bloodstream infections (CRBSI) is a source of high morbidity and mortality. Central line-associated bloodstream infection (CLABSI) occurs when a patient develops a bloodstream infection with the site of the infection being from a biofilm growth in a central venous catheter (CVC). These infections have severe consequences: 30-35% of patients die in intensive unit care with catheter-related bloodstream infections. The replacing of CVC at the bedside is the gold standard of treatment CRBSI behavior, because of the extreme tolerance towards antibiotic and the difficulty to eradicate biofilms. On the other hand, the problem of bacterial multiresistance or pan-drug resistance development in the conventional systemic treatment of CLABSI is a public health challenge that needs to be addressed.

Materials such as polyurethane, Teflon® and silicone are the most used in the manufacture of catheters given that they have low cytotoxicity, as well as lower thrombogenic and endothelial injury risk, which would theoretically be associated with lower infection rates. The reality is that in practice these polymers are highly prone to microbial adhesion, formation of biofilms and consequent development of infections associated with the catheter.

Cellular interactions with material surfaces are critical to the performance of medical devices and systems immersed in aqueous environments or are covered by an aqueous film. Much research has concerned host cell-substrate interaction of implanted medical devices; however, the interaction of bacterial cells, which in humans outnumber host cells at least 10 to 1, with the material surface is also important. The useful lifetime of biomedical implants can be greatly diminished by development of biofilms.

A biofilm is composed of bacteria, proteins, and cells that adhere and aggregate on the material surface. Biofilm development begins when a single planktonic cell attaches to an available material surface in response to environmental cues, including nutrient availability and physicochemical forces. Once adhered to the material surface, the bacteria begin to proliferate, secreting extracellular polysaccharide substance (EPS) and forming multilayer cell clusters on the material surface to create the biofilm. Biofilm formation on an implanted medical device can cause persistent infection, especially if parts of the biofilms shed off into the bloodstream, eliciting immune response and triggering the release of harmful toxins in the body. Biofilms have been reported to account for over 80% of microbial infections in humans; in fact, many of undiagnosed chronic diseases are thought be of biofilm origin.

A number of strategies based on AntiMicrobial Coatings (AMCs) of the biomaterial were proposed to overcome this problem. Many different chemical strategies and technologies for AMCs have been described: (a) AMCs may contain active eluting agents (ions or nanoparticles of silver, copper, antibiotics); (b) immobilized molecules that become active upon contact (quaternary ammonium polymers, peptides, chitosan); or (c) light-activated molecules (TiO₂ or photosensitizers).

The main problems still unresolved for these methodologies include spreading of antibiotic resistance and ion release rate that limits the effectiveness of the antimicrobial action. Furthermore, silver resistance bacterial genes were reported in the last years. Recent advances in materials and surface engineering have allowed the design of surfaces with global roughness and defined patterns. In this regard, nanotechnology offers new methods and techniques to design multiple topographic patterns with different shapes and sizes that can inhibit both bacterial adhesion and biofilm formation in various materials such as silicone, polydimethylsiloxane and polystyrene.

There are different published methodologies of processes to add microbial activity onto plastic surfaces. A general survey is synthesized in Nichols publication of Biocides in Plastics (Nichols, D. (2004). Biocides in Plastics, 15 (12), 28-39). The most conventional and oldest technique is the chemical modification approach, conventionally with silver particles thin films embedded on plastics, either varying thickness, concentration, deposition techniques (lamination, printing, coating, etc). More recently, several papers describing nanostructured surfaces with respect to their potential impact on bacterial adhesion and inhibit bacterial colonization (Ferdi Hizal et al. Staphylococcal Adhesion, Detachment and Transmission on Nanopillared Si Surfaces. ACS Applied Materials & Interfaces. DOI: 10.1021/acsami.6b09437, 2016). In these cases, studies are made on materials without application so far: Si-nanopillars on Si surfaces are fragile which makes them unsuitable for most practical clinical applications.

This type of approach is driven by the ability to control the activity without the addition of chemical compounds, only by topography and non-specific interactions. The impact of using topography instead of chemical compounds is very attractive since it does not require the addition of additional compounds to the system, which could bring different chemistry and reactivity, chemical compatibility and even toxicity, and ultimately generate bacterial resistance.

### SUMMARY OF INVENTION

In order to address and overcome at least some of the above-mentioned drawbacks of the prior art solutions, the present inventors developed an antimicrobial surface having improved features and capabilities.

Accordingly, one object of the present invention relates to an antimicrobial surface comprising a first array of nanostructures spaced apart on a support, characterized in that it comprises a second array of spaced apart nanostructures located on said support and on said first array nanostructures, said second array nanostructures having different size features compared to the nanostructures of said first array.

In one embodiment, said size features comprises one of height, width, diameter, center-to-center distance and/or geometrical arrangement.

In one embodiment, said nanostructures of said second array comprises nanodots and/or lamellar nanopatterns.

In one embodiment, said nanostructures of said second array are spaced apart by a center-to-center distance of at least 10 nm, such as for instance 50 nm.

In one embodiment, said nanostructures of said second array are spaced apart by a center-to-center distance of less than 200 nm, such as for instance 80 nm.

In one embodiment, said nanostructures of said second array have a height or amplitude comprised between 5 and 100 nm, such as for instance between 10 and 50 nm.

In one embodiment, said nanostructures of said first array comprises nanopillars or nanowalls.

In one embodiment, said nanostructures of said first array are spaced apart by a center-to-center distance of at least 100 nm.

In one embodiment, said nanostructures of said first array are spaced apart by a center-to-center distance of less than 2000 nm.

In one embodiment, said nanostructures of said first array have a height comprised between 100 and 1000 nm.

In one embodiment, said nano/micro structures of said first array have a width or diameter comprised between 100 and 1000 nm.

In one embodiment, said nanostructures of said second array and/or said first arrays comprise or consist of hydrophobic and hydrophilic portions or areas of a polymer, such as a copolymer.

In one embodiment, said copolymer is one of a linear copolymer, a branched copolymer and a grafted copolymer.

Another object of the present invention relates to an article of manufacturing comprising an antimicrobial surface according to the invention.

In one embodiment, said article of manufacturing is selected from a list comprising a contact lens, a dermal patch, a central or peripheral venous catheter, a connector, an endotracheal tube, an intrauterine device, a replacement heart valve, a transient or percutaneous pacemaker, a catheter for peritoneal dialysis, a ventricular lead, a cerebrospinal fluid shunt, a nasogastric tube, a joint prostheses, a tympanostomy tube, an urinary catheters and a stent.

The above and other objects, features and advantages of the herein presented subject-matter will become more apparent from a study of the following description with reference to the attached figures showing some preferred aspects of said subject-matter.

### DETAILED DESCRIPTION OF THE INVENTION

The subject-matter herein described will be clarified in the following by means of the following description of those aspects which are depicted in the drawings. It is however to be understood that the subject matter described in this specification is not limited to the aspects described in the following and depicted in the drawings; to the contrary, the scope of the subject-matter herein described is defined by the claims. Moreover, it is to be understood that the specific conditions or parameters described and/or shown in the following are not limiting of the subject-matter herein described, and that the terminology used herein is for the purpose of describing particular aspects by way of example only and is not intended to be limiting.

Unless otherwise defined, technical and scientific terms used herein have the same meaning as commonly understood by one of ordinary skill in the art to which this invention belongs. Further, unless otherwise required by the context, singular terms shall include pluralities and plural terms shall include the singular. The methods and techniques of the present disclosure are generally performed according to conventional methods well known in the art and as described in various general and more specific references that are cited and discussed throughout the present specification unless otherwise indicated. Further, for the sake of clarity, the use of the term “about” is herein intended to encompass a variation of +/−10% of a given value.

The following description will be better understood by means of the following definitions.

As used in the following and in the appended claims, the singular forms “a”, “an” and “the” include plural referents unless the context clearly dictates otherwise. Also, the use of “or” means “and/or” unless stated otherwise. Similarly, “comprise”, “comprises”, “comprising”, “include”, “includes” and “including” are interchangeable and not intended to be limiting. It is to be further understood that where for the description of various embodiments use is made of the term “comprising”, those skilled in the art will understand that in some specific instances, an embodiment can be alternatively described using language “consisting essentially of” or “consisting of.”

The present invention is based on the development of an antimicrobial surface based on an antibiofilm nanomaterial mechanotransduction concept: a new bifunctional nanotopography/nanopattern approach to coat the surface of items such as catheters, which on the one hand controls bacteria attachment and orientation and exert a mechanical rupture of the bacteria to produce “stretch-and-break”, and on the other hand controls the characteristic assembly and packing behaviors of plasmatic proteins adsorbed on the surface. The surface is referred as being “stretch-and-break” to the extent that it produces mechanical rupture of the bacteria that adhere to its surface, thereby reducing the proliferation of bacteria and therefore the risk of infection or illness due to the presence of bacteria. In addition, the stretching on the bacteria wall may be activating mechanosensitive channels, opening the large pore of channels inappropriately in detrimental to the cell with changes in the composition of the bacterial cytoplasm, resulting in a collapsed cell. This mechanical approach offer many advantages compared to the widely studied coatings commonly used in the art: by avoiding the use of chemical agents, the bactericidal properties will not present a decay due to depletion of the agent, no resistant strains will be generated, and there are no risks associated with the release of ions and potentially allergenic substances. Moreover, the possibility of modulating very precisely the surface topography would allow engineering of specific patterns that could cover a broader spectrum of bacteria (both Gram positive and negative).

In polymeric-based medical devices (like catheters) thrombosis and infections are strongly interrelated and therefore the present approach tackles both problems simultaneously. Bacteria interact with surfaces that are in contact with human blood, therefore the surface is primarily covered by proteins. Up to the inventors' knowledge, the inventive concept behind the invention is the first one that takes these key parameters into consideration for the design of an antimicrobial surface. Moreover, the absence of antimicrobial agents (antibiotics, peptides, silver, etc) will likely prevent the development of bacterial resistance, which is a common problem for antimicrobial agents.

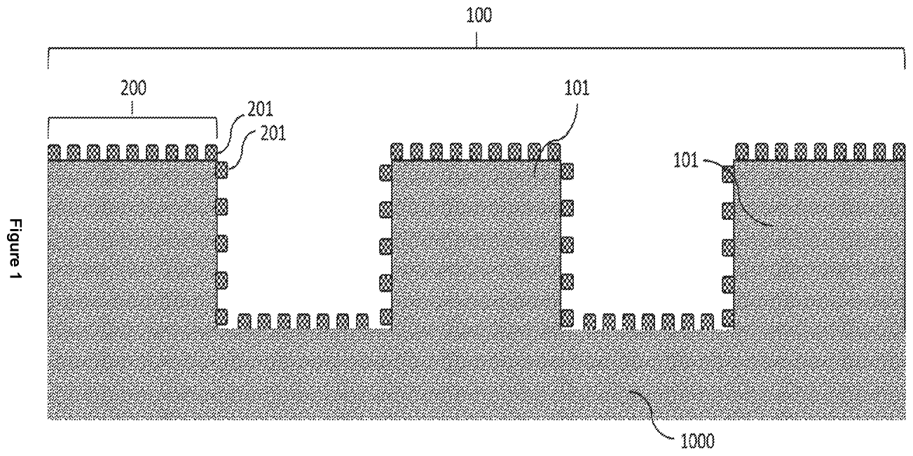

According to the invention, the antimicrobial surface of the invention comprises two patterns of nanostructures 100 and 200 located on a support surface 1000, as exemplarily shown as one embodiment in FIG. 1. According to this embodiment, the production of the antimicrobial surface of the invention is based on a bifunctional nanotopography-textured approach, which on the one hand control bacteria attachment and orientation and exert a mechanical rupture of the bacteria to produce “stretch-and-break”, and on the other control the characteristic assembly and packing behaviors of plasmatic proteins adsorbed on the surface. A prototypical surface can be advantageously considered to be the inner lumen of catheters, where the risk of microbial colonization and infections is a frequent issue.

The bigger bacteria anchorage nanostructures 101 of the first array 100 are used to exert on bacteria mechanical forces and consequent tension in membrane, thereby activating mechanosentitive channes and “stretch-and-break” mechanism, whereas the smaller nanostructures 201 of the second array 200 are used to control conditioning protein layer that plays a pivotal role in the establishment of biofilms and inter-communication among bacteria. For the sake of clarity and simplicity, only a portion of the second array 200 is indicated in FIG. 1, and it is tacitly understood that the entire surface of the support substrate 1000 is covered by the second array 200, in accordance with FIG. 1. However, in some embodiments not shown herein, only a portion or a plurality of portions of the support substrate 1000 is/are covered by the second array 200.

Exemplary features of both sets of interlaid sets of nanostructures are given below: nanostructured surfaces having bacteria anchorage nanostructures embodied as nanopillars and/or nanowalls (first array 100): Center-center: 100-1200 nm; Height: 100-1000 nm; Diameter of the nanopillars is 100 to 1000 nm; Diameter at the tips: 100-1000 nm.

Nanostructured surfaces for control conditioning protein layer (second array 200): Spacing: 30-100 nm; Amplitude: 5-100 nm. In embodiments of the invention, said nanostructures of said second array 200 comprise nanodots and/or lamellar nanopatterns.

The material used for producing the antimicrobial surface of the invention may comprise one or more of the following materials: polyurethane, PMMS, teflon or silicone.

In one implemented, non-limiting additional or alternative embodiment, to produce the nanostructured patterns the inventors have explored the use of polymers having hydrophobic and hydrophilic domains alternating in various ways, representing the “nanostructures” according to the invention; in particular, block copolymer (BCP) thin films have shown to have a good antimicrobial efficacy and low toxicity for human cells along with a high selectivity versus bacterial cells. Moreover, block copolymers with various morphologies and compositions are relatively inexpensive materials that can be deposited as thin films on surfaces, offering a versatile platform where nanotopography, dimensionality and roughness can be easily tuned. This approach can be used in combination with plastic surfaces produced by e.g. hot imprint technology to print the selected patters directly on the starting materials and provided a low-cost and scalable approach for fabricating antimicrobial surfaces.

In the smaller nanometer regime (up to about 100 nm, second array 200), the present surface configuration controls how the proteins present in human plasma bind to the polymeric surface. In embodiments of the invention, the assembly and packing of the proteins of the conditioning layer is controlled by nonspecific hydrophilic-hydrophobic interactions. The main proteins present in human plasma assemble preferentially on the hydrophobic domains. According to the invention, on hydrophilic areas the proteins either do not adsorb or adsorb with a conformation that exposes less binding sites for coagulation factors, an important parameter to avoid thrombus formation. The nm regime spacing between domains is controlled in one embodiment using block copolymers. On the hydrophobic domains, the proteins adsorb in a conformation that allows bacterial anchorage, therefore the spacing of these adsorption sites also determine how stretched is the bacterial membrane.

In the bigger nanometric regime (first array 100): this is comparable to the size of most bacteria (about 1000 nm). In this regime, the bacteria adhesion can be influenced by controlling the spacing of adhesion points (protein-bacteria) and using topography strategies to promote the spatial segregation and avoid the bacteria intercommunication, that are key parameters to inhibit biofilm formation. The deformation of the bacterial membrane to find anchorage points to the surface triggers the “stretch and break” mechanism that ultimately kill the bacteria by activating mechanosensitive channels. The spatial isolation of the bacteria inhibits the secretion of extracellular polymeric substances (EPS) making the bacteria vulnerable.

In additional or alternative embodiments of the invention, both the nanostructures of said second array 200 and of said first array 100 comprise or consist of hydrophobic and hydrophilic portions or areas of a polymer, such as a copolymer. Said copolymer can be one of a linear copolymer, a branched copolymer and a grafted copolymer.

According to particular aspects of the present invention, a method is provided of altering the surface of a venous central catheter materials made from silicones or thermoplastic-based materials. The method comprises a printed surface with the required topography. The dimensions and resolution of the required topographies are only achievable by silicon lithography procedures, therefore UV-Nanoimprint lithography is used to convert these master structures in silicon onto flexible replica stamps. To prepare the injection molding insert, the printed surface is made by inkjet based UV curable resist deposited on a conformable polymer foil and embossed with the flexible transparent replica stamp. This conformable printed foil is then thermoformed and used as flexible insert inside a catheter mold. Both arrays were found fully stable after sterilization with ethylene oxide, which is the standard sterilization procedure for medical devices

In one embodiment, an article of manufacturing comprising the surface of the invention is selected from a list comprising a contact lens, a dermal patch, a central or peripheral venous catheter, a connector, an endotracheal tube, an intrauterine device, a replacement heart valve, a transient or percutaneous pacemaker, a catheter for peritoneal dialysis, a ventricular lead, a cerebrospinal fluid shunt, a nasogastric tube, a joint prostheses, a tympanostomy tube, an urinary catheters and a stent.

### EXAMPLES

### Multi-Drug Resistant Klebsiella pneumoniae Strain

Results Bacterial Assays

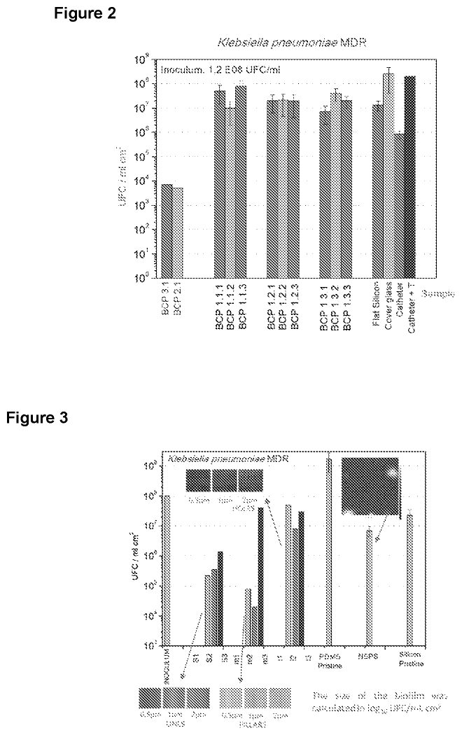

Nanostructured Substrates: Block Copolymer (FIGS. 2 & 3)
BCP 1.X.X=PS-b-PEO⇒Nanopattern DOTS
BCP 2.X=PS-b-PEO EtOH RIE⇒Nanopattern DOTS
BCP 3.X=PS-b-P2VP⇒Nanopattern LAMELLAR
PDMS: Patterns with 500 nm-2 μm range structures
   Nanopattern HOLES/PILLARS/LINES

Nanostructured Substrates:
PS-b-PEO/PS-b-P2VP
Nanostructured surfaces have a bacteria anchorage nanopillars:
   Center-center: 150-800 nm
   Height: 200-1000 nm
   Diameter of the nanocolumns is 50 to 100 nm.
   Diameter at the tips: 10-100 nm.
Nanostructured for control conditioning protein layer (Lamellar):
   Spacing: 30-60 nm
   Amplitude: 10-30 nm
Silicon flat
Cover glass
Commercial CVC (Arrow™)
BCP thin films composed of polystyrene and polyethylene oxide (PS-b-PEO), and poly (styrene-block-2-vinylpyridine) (PS-b-P2VP) with different molecular weights and reactions conditions were used for Gram-negative biofilm formation study. After 24 hour, the size of the biofilm was calculated in log10 UFC/mL cm2 showing in some cases a reduction in the quantification of viable bacteria of up to two-three orders of magnitude with respect to the unmodified material and of a commercial catheter.
We found that bacteria attachment and orientation at the single cell level was modulated by PDMS (Polydimethylsiloxane) nanostructures with nanometer scale periodic features (pillars, lines and holes in 1 μm dimension), obtained by hot imprint technology. This spatial segregation of single bacteria affects the interbacteria communication and prevents the subsequent formation of colonies and the development of a bacterial biofilm. The exposed cells became flattened and wrinkled and lost their cellular integrity, displaying perturbed bacteria membrane morphologies. Therefore, micro/nanopatterning the surface not only prevents biofilm growth but also kills the attached bacteria.
Bifunctional Nanotopography Approach:
1—We found that block copolymer thin films (mostly 2D) with well-spaced hydrophilic-hydrophobic areas by non-specific interactions control the distribution of precursors plasmatic proteins. Since the protein layer is needed for the formation of biofilms (bacterial contamination), we observe that the bacteria either do not attach, do not growth (a smaller size indicates lower cell division and metabolism) or die.
2—the pure nanotopographical (physical contrast) approach using printed polymers with 3D nano/micro features shows that the spatial segregation of single bacteria can be controlled to affect the interbacteria communication and prevent the subsequent formation of colonies and the development of a bacterial biofilm. The exposed cells became flattened and wrinkled and lost their cellular integrity, displaying perturbed bacteria membrane morphologies. Therefore, micro/nanopatterning the surface not only prevents biofilm growth but also kills the attached bacteria.
Nanostructured PRISTINE substrates: AFM characterization
   Patterns observed in BCP 1.X.X thin films
   BS 1.1.2: 150° C. in vacuum for 16 hours

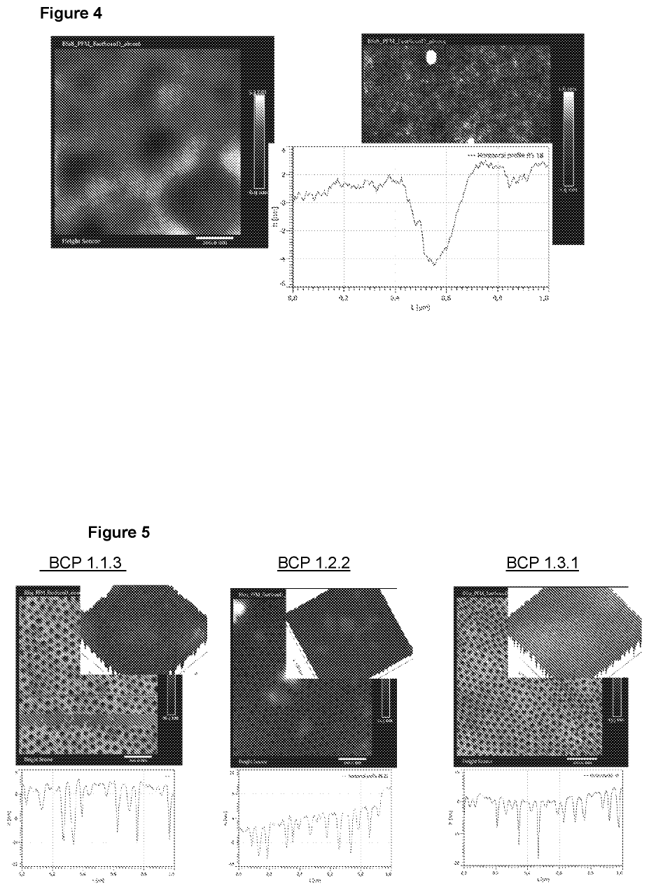

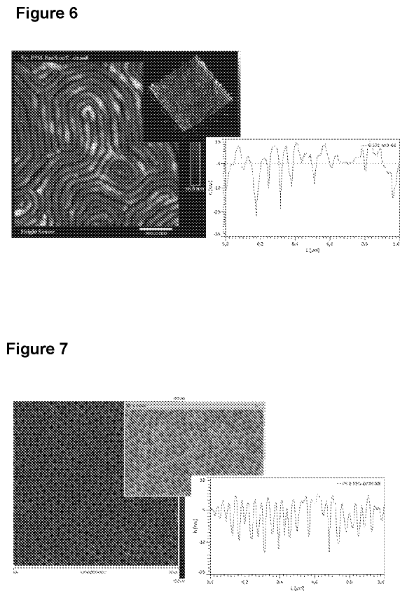

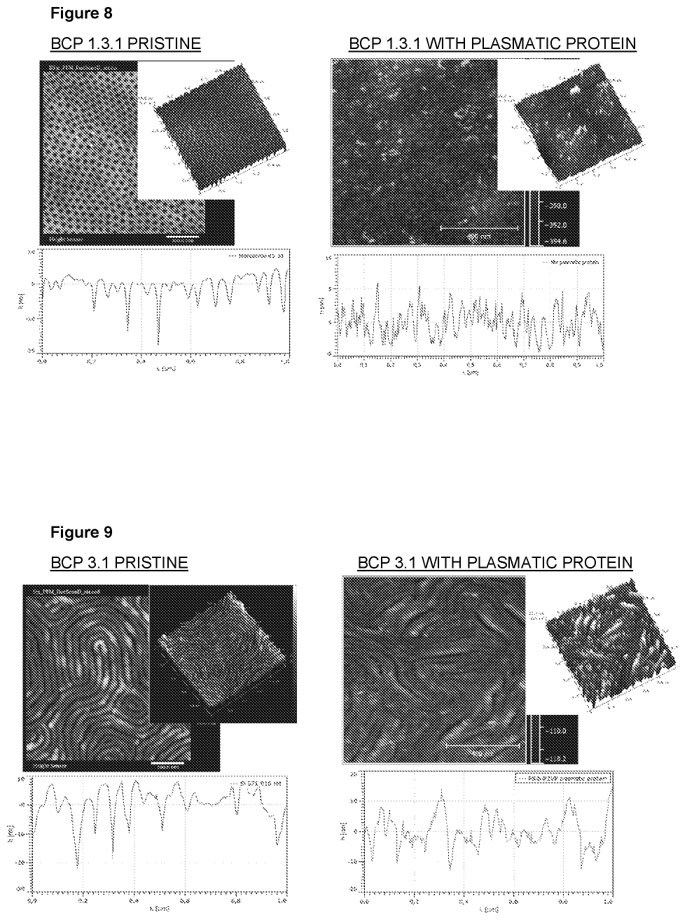

Patterns: Altered patterns, probably because no solvent annealing (FIG. 4)
Patterns observed in BCP 1.X.X. thin films
BCP 1.1.3: 0.15 ml H₂O 10′/0.3 ml THF 20′/SR˜3,4
Dot patterns Spacing: 50 nm Amplitude: 10-25 nm
BCP 1.2.2: 0.10 ml H₂O 10′/0.75 ml THF 20′/SR˜2,3
Dot patterns Spacing: 43 nm Amplitude: 7-10 nm
BS 1.3.1: 0.10 ml H₂O 20′/0.40 ml THF 160′/SR˜3,0
Dot patterns-Spacing: 40 nm (dots) Amplitude: 16 (dots)
See FIG. 5
Patterns observed in BCP 3.X. thin films
BCP 3.1 (FIG. 6): Substrate with a PS-b-P2VP BCP thin film on top.
Patterns—Spacing: 43 nm. Amplitude: 15-30 nm
Patterns observed in BCP 2.X thin films (AFM)
BCP 2.1 (FIG. 7): PS-b-PEO+EtOH RIE silicon substrate
Patterns—Spacing: 45 nm. Amplitude: 15-20 nm
Nanostructured substrates WITH PLASMATIC PROTEINS: AFM characterization (FIG. 8).

See FIG. 9 that shows PCP 3.1. Pristine and with plasmatic protein

When a surface is exposed to a liquid containing bacteria in planktonic state (e.g. blood plasma or culture medium), it triggers a series of processes that prepare the exposed surface for its colonization and biofilm formation. A conditioning layer is form with proteins that are able to deposit onto the surface and will serve as attaching sites for the bacteria. Proteins with small hydrodynamic drag and high concentration are deposited first. Then, adsorption of different proteins, as well as their final conformation and spatial distribution over the surface, will depend on the individual chemical affinity with the substrate, its thermostability, and variables such as electrostatic interactions and local flow properties. As a result, inhomogeneous protein distribution might arise. Interestingly, several studies have also connected the conditioning layer building processes with thrombosis. In our approach, we control the spatial distribution of the conditioning layer, by confining it in a way that it reduces the number of available attaching sites to bacteria. When the topography and chemical characteristics of the surface are controlled at the nanoscale, it is possible to promote protein deposition and conditioning layer formation in the deeper spots (valleys). Furthermore, adsorbed proteins appear to fold in a compact shape due to confinement, thereby making the interacting sites less likely to be exposed. Our results also show that the opposite effect (enhanced biofilm) is obtained when proteins target the convex higher parts (ridges), creating an elevated and inhomogeneous layer, but large and stable enough to provide bacteria with sufficient attaching-stable sites.
SEM: Biofilms of Klebsiella pneumoniae were grown under static conditions.
   BCP 3.1.
   Morphology bacteria: Rod-shaped.
   Size cells: 0.5-1.0 μm.
   The size of the biofilm was calculated in log₁₀ UFC/mL cm²⇒
      Inoculum: 1, 3×10⁷ UFC/ml cm²
      BCP 3.1 (24 h culture): 7×10³ UFC/ml cm²
   The assay showed that the viable cell numbers on all nanostructured surfaces significantly decreased 3 orders of magnitude compared to that on the flat surfaces.
   Bacteria numbers on the samples remained at a low level (i.e., not increasing from 24 to 48 hours), indicating effective inhibition of Klebsiella growth.
   K. pneumoniae on BCP 3.1 after 48 h under static culture
   BCP 3.1: PS-b-P2VP. Image shows damaged cells.

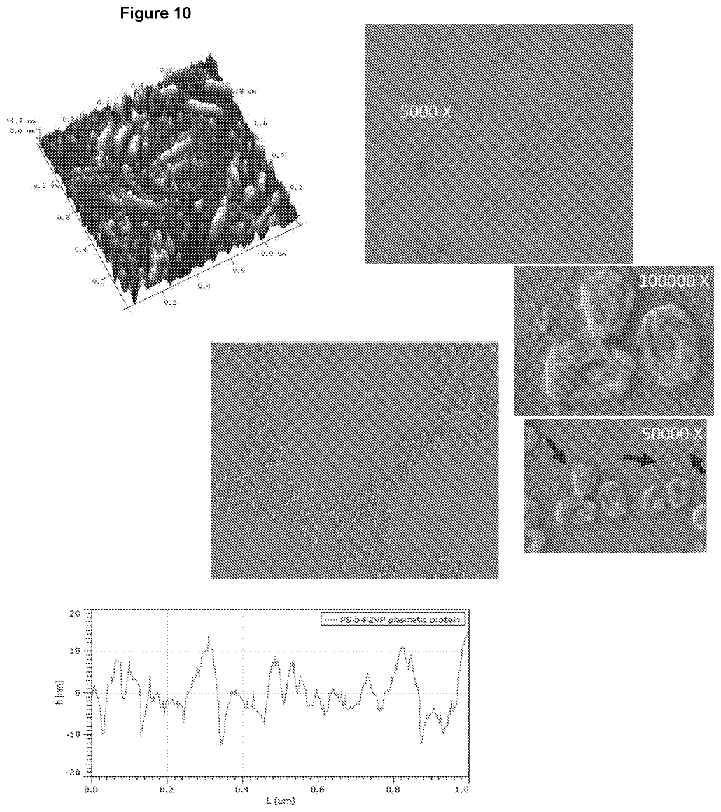

FIG. 10 shows a SEM analysis of the surface of the K. pneumoniae isolates revealed morphological changes of the bacterial cell surface. At 50000× and 100000×: shows a selection of SEM images highlighting destruction of cell membranes. Increased permeabilization of the outer membrane may explain the leakage of cytoplasmic material (arrows).

Control silicon: edge (flat) surface: Image shows healthy (turgid) bacterial cells

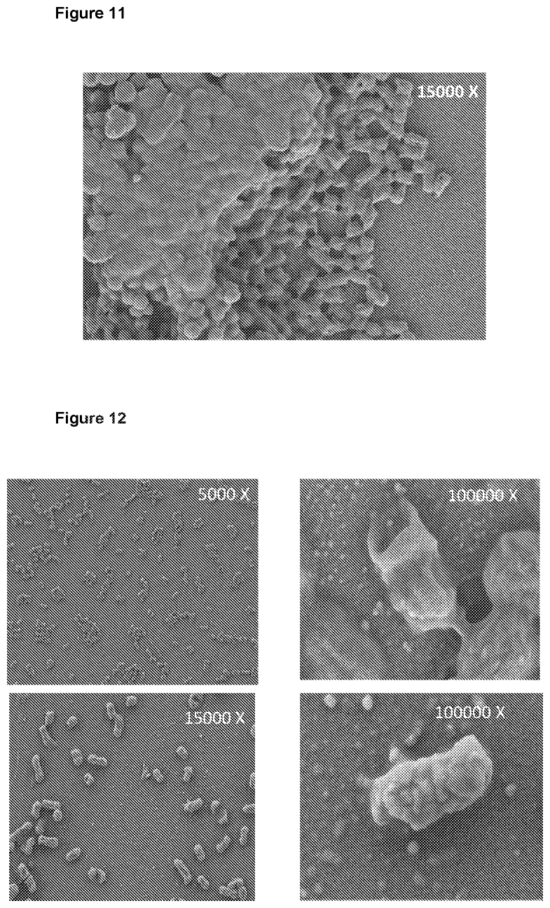

See FIG. 11

Abundant multi-layered K. pneumoniae: In the control biofilm, the cells formed a dense aggregates and EPS on the edge of the sample. It is evident that more cells attached to pristine surface compared to nanostructured polymer and they retain road-shaped form.
BCP 2.1
The surface not only prevents biofilm growth through spatial segregation of single bacteria but also inhibit extracellular polymeric substances (EPS) and hypermucoviscous of hypervirulent strains secretions
See FIG. 12

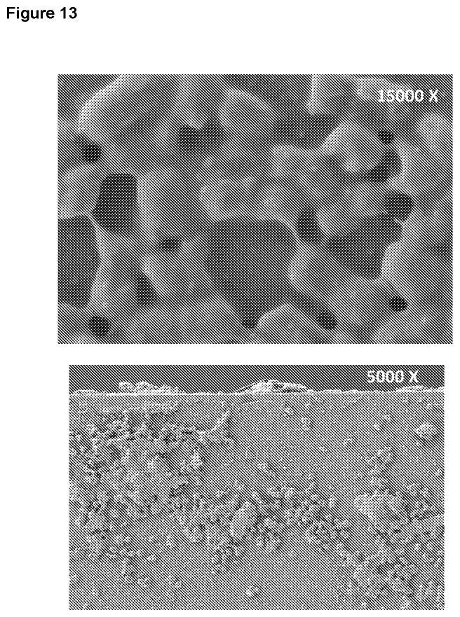

Observation at high magnification showed great alterations of bacteria membrane with collapsed and lysed cells compared to normal cells. The formation of pits was indicative of increased cell permeability due to the pronounced disruption of the structural integrity of the bacterial outer membrane that could result in potential leakage of cytoplasmic material. The irregular (lumpy) sacculi of bacteria on nanostructure indicate that the cells have been ruptured and the turgor pressure has been lost.
SEM: biofilm formation K. pneumoniae (24 h culture)
Control silicon: edge of the surface (flat)
See FIG. 13

On a non-nanopattern silicone substrate, the strains of Klebsiella pneumoniae produce a hypercapsule (hypermucoviscosa), which consists of a bacterial mucosal exopolysaccharide coating that is more robust than that of the typical capsule. Observation at 15000× magnification of these cell-associated bacterial clusters showed bacteria connected to each other by a fibrillar network composed by flexible pili that extended several nm away from the bacteria.

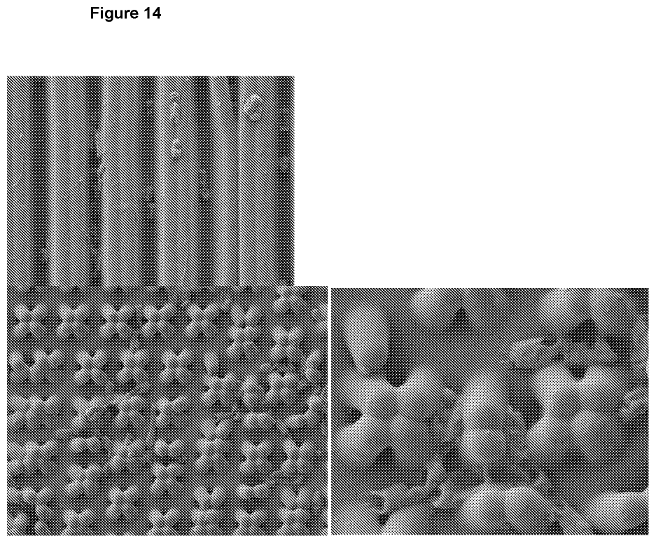

This strain built large three-dimensional colonies containing large numbers of bacteria sitting on the surface of pristine silicon. EPS formation is obviously perceived.
PDMS: LINES/PILLARS 1 μm
See FIG. 14

The exposed cells became flattened and wrinkled and lost their cellular integrity, displaying perturbed bacteria membrane morphologies.

### CONCLUSIONS

SEM: biofilm formation K. pneumoniae

On selected nanostructures, SEM analysis of the surface of the K. pneumoniae isolates revealed morphological changes of the bacterial cell surface. Observation at high magnification showed great alterations of bacteria membrane with collapsed and lysed cells compared to normal cells. The formation of pits was indicative of increased cell permeability due to the pronounced disruption of the structural integrity of the bacterial outer membrane that could result in potential leakage of cytoplasmic material (blue arrows). In addition, on these surfaces the bacteria are isolated and do not make contact with neighboring cells. These could be an important factor that resulted in the reduction of biofilm formation. The ability of these bacteria to associate into communities in biofilms is central to their pathogenicity as they confer protection from antimicrobial agents and bactericidal molecules present on host tissues.

On a non-modified substrate, the bacteria formed a dense aggregate. Key to the formation of biofilms is the release of extracellular polymeric substances (EPS). SEM pictures clearly show cell shape and the presence of extra cellular adhesive structures (exopolysaccharides and proteinaceous adhesins). Moreover, the strains of Klebsiella pneumoniae produce a hypercapsule (hypermucoviscosa), which consists of a bacterial mucosal exopolysaccharide coating that increases resistance to neutrophil phagocytosis in vitro. Resistance to some antibiotics in K. pneumoniae is hypothesized to be due to increased capsular polysaccharide production. Observation at 15000× magnification of these cell-associated bacterial clusters showed bacteria connected to each other by a fibrillar network composed by flexible pili that extended several nm away from the bacteria.

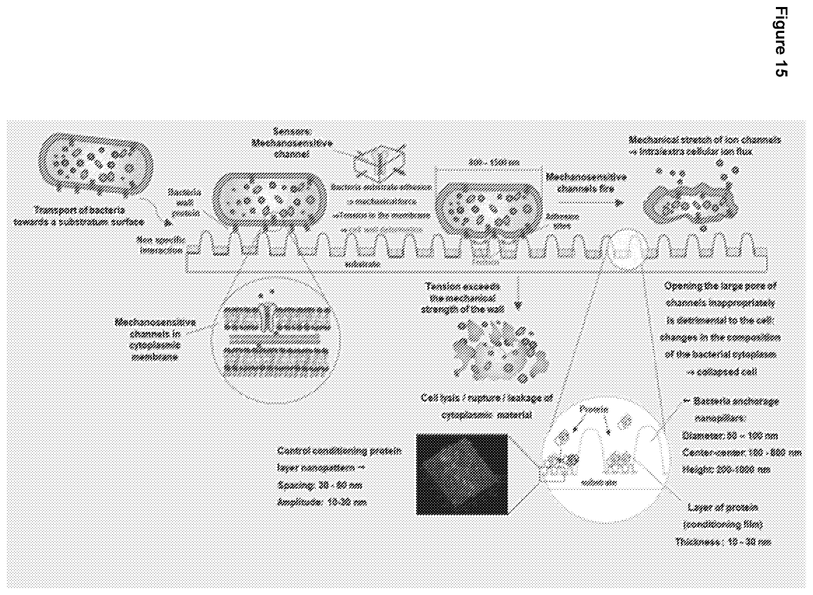

See FIG. 15
Bifunctional nanotopography approach to coat the surface of catheters, which on the one hand controls bacteria attachment and orientation and exert a mechanical rupture of the bacteria to produce “stretch-and-break”, and on the other controls the characteristic assembly and packing behaviors of plasmatic proteins adsorbed on the catheter. Two effects have been compared using the same substrate: mechanical effects and control of protein adsorption.

DISRUPT THE CELL MORPHOLOGY: The surface is referred as being “stretch-and-break” to the extent that to produce mechanical rupture of the bacteria to adhere to the surface thereby reducing the proliferation of bacteria, reducing the risk of infection or illness due to the presence of bacteria. In addition, the stretching on the bacteria wall may be activating mechanosensitive channels, opening the large pore of channels inappropriately in detrimental to the cell with changes in the composition of the bacterial cytoplasm, resulting in a collapsed cell. This mechanical approach offer many advantages compared to the widely studied coatings in literature: by avoiding, the use of chemical agents the bactericidal properties will not present a decay due to depletion of the agent, no resistant strains will be generated, and there are no risks associated with the release of ions and potentially allergenic substances.
The distance between nanospikes exert on bacterial a mechanical force which play a pivotal role in regulating bacteria fate. From a perspective of nanotechnology, nanopatterns materials are an appealing option for mechanotransduction due to their capabilities in manipulation of mechanical forces via surface characteristics. Because the bacteria have access to a very limited number of anchorage points, for its rigidity and size only allows them to come into contact with the upper area of a limited number of nanopillars to develop adhesion points and consequent tension in membrane, mechanosentitive channel activation and “stretch-and-break”. This membrane integrity disruption leads to a reduced ability to control the movement of substances in and out of a bacterial cell, causing cell death. In this manner, mechanotransduction relies on bacterial mechanoreceptors to convert mechanical stimuli into biochemical signals, which can induce different cell responses including proliferation or death.
Micro/nanopatterning the surface not only prevents biofilm growth through spatial segregation of single bacteria but also inhibit extracellular polymeric substances (EPS) and hypermucoviscous of hypervirulent strains secretions in Gram negative bacteria, which would provide vulnerability to the action of antibiotics and cells of immune system.
Nanostructured surfaces supported few viable bacteria compared to flat surfaces.
Nanopatterns are effective to prevent formation of bacterial aggregates (this is a precursor of biofilm formation). REDUCTION IN CELL AGGREGATES.
The confinement of plasmatic proteins on the nanopattern decreases the propensity to form thrombus on the catheter (there is strong evidence that catheter-related thrombosis and infection are interrelated and should therefore not be seen as separate entities).

The major complications associated with intravascular catheters include thrombosis and infections. It is important to remark that the surface of biomaterials is instantaneously covered by either a layer of glycoproteins when exposed to body fluids in vivo, or to glycoproteins containing culture media in vitro. Since the blood-catheter interface triggers a complex series of events including protein adsorption, adhesion and activation of platelets and leukocytes, complement activation and coagulation, the physicochemical properties of the catheter material such as surface wettability and roughness may influence the propensity of thrombosis. Thus, there is strong evidence that catheter-related thrombosis and infection are interrelated and should therefore not be seen as separate entities.

Therefore, to have a more realistic model we decided to study the characteristic assembly and packing behaviors of proteins—that plays an important role in blood clotting—on the nanopatterns. The interplay between hydrophobic and electrostatic interactions existing between the protein and the polymeric segments as well as the electrostatic repulsion between the proteins can lead to either elongated or globular protein conformations. We found on adsorbed proteins conformational changes can either expose or hide binding sites, thus turning them on or off and a closed compact conformation is necessary to prevent aberrant interactions among protomers and between proteins and cell surface receptors and such macromolecules. The different conformation of the initial protein layer will likely affect the formation of bacterial films on the BCP samples or to avoid unwanted blood clotting on implant materials and therefore it is important to have a clear picture of the roughness and conformation of glycoproteins on the surface. The protein adsorption and distribution under physiological conditions of the films previous to exposure to bacterial growth to understand the morphological and chemical mechanisms that ultimately determine low bacterial adhesion and/or membrane cell disruption.

### Antibiotic Resistance

The disc diffusion method is based on the presence or absence of a zone of growth inhibition, which is measured in millimeters. The interpretation of the test is based on the correlation between the diameter of the zone of inhibition (mm) with the MIC (μg/mL) for each antimicrobial and microorganism. This test is done on the isolated strains not on the antimicrobial surfaces.

Detection of antibiotic resistance was performed with the set of 19 antibiotics: aztreonam (30 μg), cefotaxime/clavulanic acid (30/10 μg), amikacin (30 μg), ceftriaxone (30 μg), ceftazidime (30 μg), ceftazidime/clavulanic acid (30/10 μg), cefotaxime (30 μg), cefepime (30 μg), cefoxitin (30 μg), colistin (10 μg), Sulbactam/ampicillin (20 μg), 1:1 trimethoprim 1.25 μg+sulphamethoxazole 23.75 μg, ciprofloxacin (5 μg), levofloxacin (5 μg), gentamicin (10 μg), imipenem (10 μg), ertapenem (10 μg), meropenem (10 μg), piperacillin+tazobactam (110 μg) and ampicilin (10 μg).

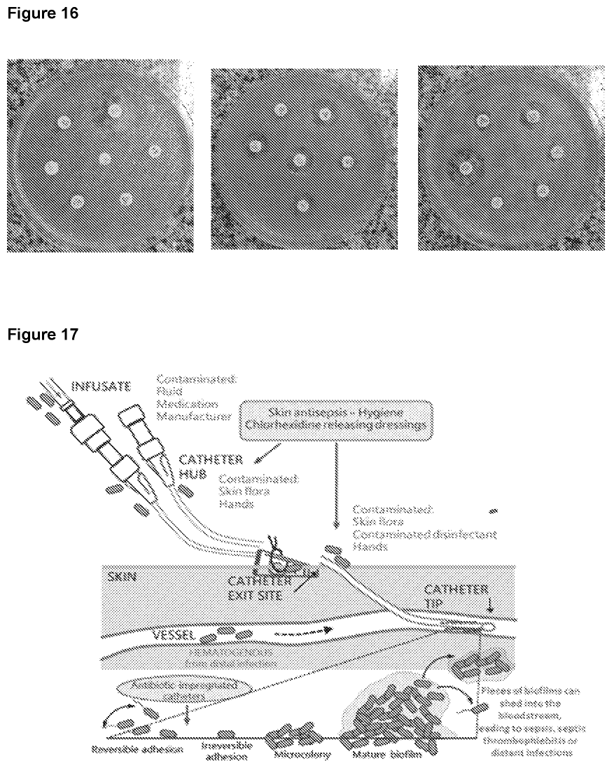

See FIG. 16

The strain presents:
Extended-spectrum β-lactamase
Aztreonam≤27 mm
Cefotaxime≤27 mm
Ceftazidime≤22 mm
Ceftriaxone≤25 mm
Increase of ≥5 mm in the diameter cefotaxime/clavulanic acid
   AmpC β-lactamase
   Carbapenemase Metallo-β-lactamase

Model biofilm development may be subdivided into the following steps:
(1) reversible attachment of the microorganism to the surface characterized by non-specific interactions where cells are easily removed by gentle rinse.
(2) irreversible attachment active mechanisms as pili (or fimbriae), adhesion proteins, and exopolymers contribute to a stronger adhesion to the surface through molecular-specific interactions.
(3) cell-cell adhesion and proliferation bacterial colonies
(4) maturation of the biofilm containing an additional polymer matrix, which stabilizes the biofilm against fluctuations.
(5) detachment of cells

the bactericidal efficiency of the reference and nanostructured surfaces with an area of 1×1 cm2

was quantitatively assessed and compared using a proliferation assay.

See FIG. 17

### Bacterial Preparation

Clinical isolated of Klebsiella pneumoniae was obtained from patient hospitalized at the San Justo Children's Hospital—Argentina in March 2018 isolated from the catheter tip culture and from two blood samples, drawn from the catheter before removal and from a central vein (microbiologically confirmed catheter-related bloodstream infection). The samples were processed and identified by the standard microbiological protocols and procedures.

Antimicrobial Susceptibility Disk Diffusion Test:

The suspension for inoculum was prepared from 6 isolated colonies and turbidity was compared with 0.5 Mc Farland standard. Sterile cotton swab was soaked in this suspension was used to make lawn culture on Muller Hinton (CLSI) agar plates.

### Long-Term Biofilm Formation Under Static Conditions

A Klebsiella pneumoniae strain was grown on Levine EMB agar (Britania, Argentina) at 37° C. Bacterial inocula were prepared in 10 mL of Brain Heart Infusion (BHI, Britania) by inoculating a 2 colonies from the Levine agar plate and culturing overnight at 37° C. Afterwards, the bacterial suspension was adjusted to 10⁸ colony forming units (CFU)/mL in fresh growth medium (0.5 Mc Farland standard) and was used immediately for the inoculation of samples. The CFU was confirmed by viable count after the inoculation.

The nanostructured and control substrates were placed on P-60 nutritive agar Columbia plates and 200 μL of bacterial suspension was seeded onto each substrate and culturing for 24 h at 37° C. to allow biofilm formation. The substrates with biofilms were then removed and were washed with sterile Phosphate buffered saline (1×) in order to remove or detach planktonic cells.

The attached bacteria or biomass on nanostructured and control substrates was quantified using a multi-step process of cell removal, serial dilution method and plate counting for viable cell counts. Samples were individually placed in Falcon tubes containing 2 mL of sterile phosphate-buffered saline and incubated at room temperature for 30 min to promote biofilm disassembly. The irreversibly adherent bacteria were detached by vortex for 10 min. The number of bacteria in the suspension was then determined by serial dilution followed by bacterial culture on p-100 nutrient agar Columbia plates. The plates were incubated at 37 C overnight prior to colony counting. A triplicate was carried out in each case. The number of colonies forming units was assumed to be equivalent to the number of viable cells in suspension.

### SEM

To visualize the bacteria by SEM following the corresponding time of incubation, the bacteria were fixed onto the surface by immersion in 2.5% glutaraldehyde solution (Sigma Aldrich) in 0.1 M potassium phosphate buffer (potassium phosphate monobasic and potassium phosphate dibasic, pH 7.2, Sigma Aldrich) for 24 h at room temperature. The surfaces were then dehydrated by sequential immersion in 20%, 40%, 60%, 80% and 100% ethanol for 10 min each. The samples were air dried, mounted onto sample holder, and sputtered with gold before being viewed by SEM.

While the invention has been disclosed with reference to certain preferred embodiments, numerous modifications, alterations, and changes to the described embodiments, and equivalents thereof, are possible without departing from the sphere and scope of the invention. Accordingly, it is intended that the invention not be limited to the described embodiments and be given the broadest reasonable interpretation in accordance with the language of the appended claims.

1-16. (canceled)

17. An antimicrobial surface comprising:
a first array of nanostructures spaced apart on a support,
wherein the first array is interlaid by a second array of spaced apart nanostructures located on the support having differently-sized features compared to the nanostructures of the first array.

18. The antimicrobial surface of claim 17, wherein the differently-sized features include at least one of a height, a width, a diameter, a center-to-center distance and/or geometrical arrangement.

19. The antimicrobial surface of claim 17, wherein the nanostructures of the second array include at least one of nanodots and/or lamellar nanopatterns.

20. The antimicrobial surface of claim 19, wherein the nanostructures are spaced apart by a center-to-center distance of at least 10 nm.

21. The antimicrobial surface of claim 19, wherein the nanostructures are spaced apart by a center-to-center distance of less than 200 nm.

22. The antimicrobial surface of claim 19, wherein the nanostructures have a height or amplitude between 5 nm and 100 nm.

23. The antimicrobial surface of claim 19, wherein the nanostructures have a width or diameter between 10 nm and 100 nm.

24. The antimicrobial surface of claim 17, wherein the nanostructures of the first array include at least one nanopillars and/or nanowalls.

25. The antimicrobial surface of claim 24, wherein the nanopillars or nanowalls are spaced apart by a center-to-center distance of at least 100 nm.

26. The antimicrobial surface of claim 24, wherein the nanopillars or nanowalls are spaced apart by a center-to-center distance of less than 2000 nm.

27. The antimicrobial surface of claim 24, wherein the nanopillars or nanowalls have a height between 100 nm and 1000 nm.

28. The antimicrobial surface of claim 24, wherein the nanopillars or nanowalls have a width or diameter comprised between 100 nm and 1000 nm.

29. The antimicrobial surface of claim 17, wherein the nanostructures of the first array and/or of the second array include hydrophobic and hydrophilic portions or domains of a polymer.

30. The antimicrobial surface of claim 29, wherein the polymer is a copolymer.

31. The antimicrobial surface of claim 30, wherein the copolymer includes at least one of a linear copolymer, a branched copolymer, and/or a grafted copolymer.

32. An article of manufacturing comprising an antimicrobial surface, the antimicrobial surface including:
a first array of nanostructures spaced apart on a support,
wherein the first array is interlaid by a second array of spaced apart nanostructures located on the support having differently-sized features compared to the nanostructures of the first array.

33. The article of manufacturing of claim 32, the article including at least one from a list comprising a contact lens, a dermal patch, a central or peripheral venous catheter, a connector, an endotracheal tube, an intrauterine device, a replacement heart valve, a transient or percutaneous pacemaker, a catheter for peritoneal dialysis, a ventricular lead, a cerebrospinal fluid shunt, a nasogastric tube, a joint prosthesis, a tympanostomy tube, an urinary catheters and a stent.
